(19) United States
(12) Patent Application Publication
(10) Pub. No.: US 2013/0129047 A1
(43) Pub. Date: May 23, 2013

(54) PATIENT TABLE AND X-RAY IMAGING SYSTEM HAVING THE SAME

(71) Applicant: SAMSUNG ELECTRONICS CO., LTD., Gyeonggi-do (KR)

(72) Inventor: Jae Hyun LIM, Gyeonggi-do (KR)

(73) Assignee: SAMSUNG ELECTRONICS CO., LTD., Gyeonggi-do (KR)

(21) Appl. No.: 13/678,980

(22) Filed: Nov. 16, 2012

(30) Foreign Application Priority Data
Nov. 21, 2011 (KR) 10-2011-0121633

(51) Int. Cl. A61B 6/04

(52) U.S. Cl. CPC A61B 6/04
USPC 378/62; 5/601

(57) ABSTRACT

A patient table is capable of performing a patient examination using an imaging method in a rapid manner by reducing the time to prepare for the imaging and having a reduced manufacturing cost, and an X-ray imaging system has the patient table, in which the patient table includes a table top including material that allows radiated rays to pass therethrough, a support portion to support the table top, a mount portion to have a part of a body of a patient disposed thereon, and a stand assembly coupled to the mount portion.

### CLAIM OF PRIORITY

This application claims, pursuant to 35 U.S.C. 119(a), priority to and the benefit of the earlier filing date of a Korean patent application assigned Korean Patent Application No. 2011-0121633, filed on Nov. 21, 2011 in the Korean Intellectual Property Office, the disclosure of which is incorporated herein by reference.

### BACKGROUND OF THE INVENTION

1. Field of the Invention

The present invention relates to a patient table and an X-ray imaging system having the same, and more particularly, to a patient table provided with a mount portion that is available to be withdrawn from a table top, and an X-ray imaging system having the same.

2. Description of the Related Art

In general, an X-ray imaging system used in the medical field is designed to obtain an internal image of a human body by use of X-rays. The X-ray imaging system obtains an internal image of a human body by projecting X-rays toward a desired region of the human body, such as the head or the breast, and by detecting the X-rays passing through the desired region.

The X-ray imaging system includes an X-ray tube to project X-rays to a region of the human body, a high voltage generator to generate a high voltage that is required for generating the X-rays, and a moving apparatus to move a projecting position and a projecting direction of the X-rays. In addition, the X-ray imaging system is provided with a manipulating apparatus configured for an operator to manipulate the control of the apparatuses of the X-ray imaging system.

The X-ray imaging system includes an X-ray receiving portion that receives an X-ray and changes the received X-ray into a digital signal to transmit digital data to a personal computer. The X-ray receiving portion may be installed on a stand that is configured to obtain an X-ray image while having a patient in a standing position, or a patient table that is configured to obtain an X-ray image while having a patient in a lying position. The X-ray receiving portion may be provided in a fixed state, or in other embodiments in the prior art, detachably provided for the use of both of the stand or the patient table.

The patient table of the prior art includes a table top on which a patient lies. In order to obtain an X-ray image of a selected region of the patient, for example, the knees of the patient, an additional subsidiary apparatus is needed. When the subsidiary apparatus is used, the time to prepare for the imaging is increased, and a space for storing the subsidiary apparatus is needed. In addition, the subsidiary apparatus is not on the market and thus needs to be manufactured on demand.

### SUMMARY OF THE INVENTION

Therefore, it is an aspect of the present invention to provide a patient table enabling a subsidiary apparatus to be integrally formed therewith when an X-ray image is obtained, and an X-ray imaging system having the same.

Additional aspects of the present invention will be set forth in part in the description which follows and, in part, will be obvious from the description, or may be learned by practice of the present invention.

In accordance with one aspect of the present invention, a patient table includes a table top, a support portion, a mount portion and a stand portion. The table top may include material that allows radiated rays to pass therethrough. The support portion may support the table top. The mount portion may be coupled to the table top and have a part of a body of a patient disposed thereon. The stand portion may be coupled to the mount portion.

The patient table may further include an adjusting unit to adjust a coupling angle between the mount portion and the table top.

The adjusting unit may be coupled to a joint between the table top and the mount portion.

The adjusting unit may include a body portion coupled to the joint and a head portion coupled to the body portion. The body portion may be provided with at least one installation groove portion and the head portion may be provided with at least one protrusion portion inserted into the at least one installation groove portion.

The patient table may further include a releasing state at which the body portion is spaced apart from the head portion such that the coupling angle between the mount portion and the table top is adjusted, and a locking state at which the body portion is coupled to the head portion such that the mount portion is fixed.

The mount portion may pivot clockwise or counterclockwise to be folded to a lower side of the table top.

The mount portion may include an accommodating portion to receive the stand portion, and a damper portion to prevent the stand portion from being inclined at a predetermined angle or above.

The stand portion may be folded while pivoting counterclockwise or clockwise.

The stand portion may include a coupling portion that is coupled to the mount portion.

The coupling portion may be coupled at both sides of the mount portion and comprises an extension portion that extends from the coupling portion coupled at the both sides of the mount portion.

The extension portion may be provided at an upper side of the mount portion while being positioned to face the mount portion.

The extension portion may be positioned at one portion of the mount portion as an integral body with the mount portion.

The stand portion may have an X-ray receiving portion positioned thereon to receive X-rays.

The table top may include an assembling portion, which is provided at both sides of the table top, and to which the adjusting unit is coupled as a joint of the adjusting unit passes through the assembling portion.

The table top may further include a frame portion formed at an edge of the table top to reinforce the strength of the table top.

The mount portion may be coupled to the frame portion.

The mount portion may include a carbon plate, fabrics, and wood.

In accordance with another aspect of the present invention, an X-ray imaging system includes a patient table, an imaging apparatus, and an X-ray receiving portion. The patient table may include a table top including material that allows radiated rays to pass therethrough, a mount portion coupled to the table top, and a stand portion coupled to the mount portion. The imaging apparatus may be configured to obtain an image of an object by generating X-rays and projecting the X-rays toward the patient table. The X-ray receiving portion may receive the X-rays projected from the imaging apparatus.

The X-ray receiving portion may be disposed on the stand portion of the patient table, and the stand portion may be fixed while forming a right angle with respect to the mount portion to receive the X-rays from the imaging apparatus.

The imaging apparatus may be available to pivot to project the X-rays toward the X-ray receiving portion.

The patient table may further include an adjusting unit configured to adjust a coupling angle between the mount portion and the table top. The adjusting portion may include a body portion coupled to a joint between the table top and the mount portion and a head portion coupled to the body portion. The body portion may be provided with at least one installation groove portion and the head portion may be provided with at least one protrusion portion inserted into the at least one installation groove portion.

The patient table may include a releasing state at which the body portion is spaced apart from the head portion such that the coupling angle between the mount portion and the table top is adjusted, and a locking state at which the body portion is coupled to the head portion such that the mount portion is fixed.

The mount portion of the patient table may pivot clockwise or counterclockwise to be folded to a lower side of the table top.

The mount portion of the patient table may include an accommodating portion to receive the stand portion, and a damper portion to prevent the stand portion from being inclined at a predetermined angle or above.

The stand portion may be folded while pivoting counterclockwise or clockwise.

The table top may further include a frame portion formed at an edge of the table top to reinforce the strength of the table top, and the frame portion may include a patient handle portion detachably provided on the frame portion.

As described above, the operator may easily have the patient take a desired position when an X-ray image is obtained, so that the time to prepare for the imaging is reduced, and the examination is performed in a rapid manner. In addition, the subsidiary apparatus does not need to be stored additionally and does not need to be manufactured separately, so that the storage and space requirements as well as the manufacturing cost of the present invention are reduced.

### BRIEF DESCRIPTION OF THE DRAWINGS

These and/or other aspects of the present invention will become apparent and more readily appreciated from the following description of the embodiments, taken in conjunction with the accompanying drawings of which:

FIG. 1 is a view illustrating the principal configuration of an X-ray imaging system in accordance with an exemplary embodiment of the present invention.

FIG. 2 is an underside perspective view illustrating a folding state of a patient table in accordance with the exemplary embodiment of the present invention.

FIG. 3 is a top perspective view illustrating an unfolding state of a patient table in accordance with the exemplary embodiment of the present invention.

FIG. 4 is an exploded view with parts separated illustrating an adjusting unit in accordance with the exemplary embodiment of the present invention.

FIG. 5 is a see-through view illustrating a locking state of an adjusting unit in accordance with the exemplary embodiment of the present invention.

FIG. 6 is a view illustrating a releasing state of an adjusting unit in accordance with the exemplary embodiment of the present invention.

FIG. 7 is a view illustrating an unfolding state of a stand portion of a patient table in accordance with an alternative exemplary embodiment of the present invention.

FIG. 8 is a view illustrating a folding state of a stand portion of a patient table in accordance with the alternative exemplary embodiment of the present invention of FIG. 7.

FIG. 9 is a view illustrating an unfolding state of a patient table in accordance with still another alternative exemplary embodiment of the present invention.

FIG. 10 is an enlarged view illustrating an assembling portion of a patient table in accordance with the alternative exemplary embodiment of the present invention in FIG. 9.

### DETAILED DESCRIPTION OF THE INVENTION

Reference will now be made in detail to the preferred embodiments of the present invention, examples of which are illustrated in the accompanying drawings, wherein like reference numerals refer to like elements throughout. In the following description, a detailed explanation of known related functions and constructions may be omitted to avoid unnecessarily obscuring the subject matter of the present invention. The present invention may, however, be embodied in many different forms and should not be construed as limited to the exemplary embodiments set forth herein. Also, terms described herein, which are defined considering the functions of the present invention, may be implemented differently depending on user and operator's intention and practice. Therefore, the terms should be understood on the basis of the disclosure throughout the specification. The principles and features of this invention may be employed in varied and numerous embodiments without departing from the scope of the invention.

Furthermore, although the drawings represent exemplary embodiments of the invention, the drawings are not necessarily to scale and certain features may be exaggerated or omitted in order to more clearly illustrate and explain the present invention.

Although a patient table in according to the disclosed aspects of the present invention may be applied to various types of X-ray imaging systems, the following description is made in relation to, for example, a ceiling-mounted type X-ray imaging system, for illustrative purposed only, that has a guide rail installed on the ceiling of an examination room.

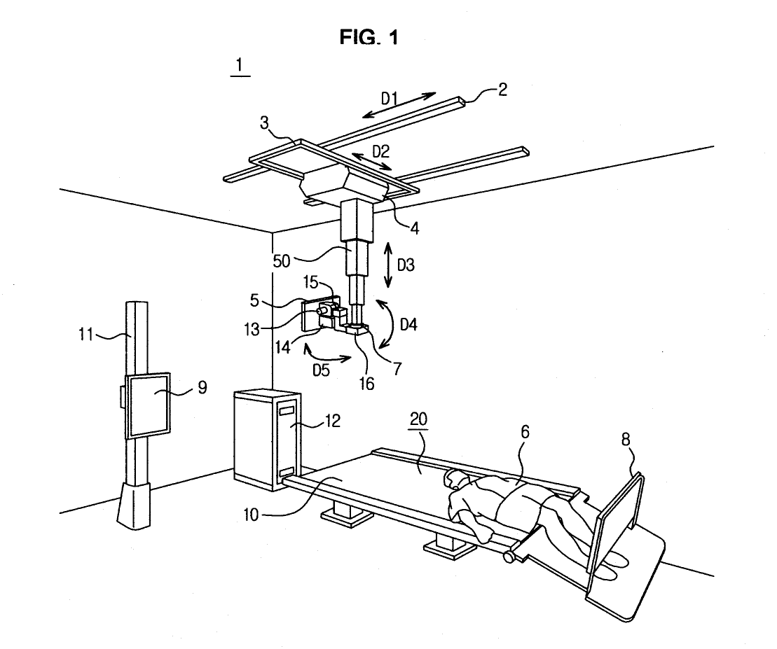

FIG. 1 is a view illustrating the principal configuration of an X-ray imaging system in accordance with an exemplary embodiment of the present invention.

Referring to FIG. 1, an X-ray imaging system 1 includes guide rails 2 and 3, a moving carriage 4, a post frame 50, an imaging apparatus 5, and a driving apparatus (not shown).

The guide rails 2 and 3, the moving carriage 4, and the post frame 50 are provided to move the imaging apparatus 5 toward, away from, and around a patient 6 and/or portions of the body of the patient 6.

The guide rails 2 and 3 include a first guide rail 2 and a second guide rail 3. The first guide rail 2 and the second guide rail 3 extend in directions perpendicular to each other.

The first guide rail 2 is installed on a ceiling of an examination room on which the second guide rail 3 and moving carriage 4 of the X-ray imaging system 1 are disposed in a slideable manner. The second guide rail 3 is disposed at a lower side of the first guide rail 2 while being installed in a slideable manner on the first guide rail 2. The first guide rail 2 is provided with a roller (not shown) installed thereto that is movable along the first guide rail 2. The second guide rail 3 moves along the first guide rail 2 while being connected to the roller.

The direction to which the first guide rail 2 extends is defined as a first direction D1, and the direction to which the second guide rail 3 extends is defined as a second direction D2. Accordingly, the first direction D1 and the second direction D2 are parallel to at least a planar portion of the ceiling or floor of the examination room while being perpendicular to each other, with D1 and D2 being substantially parallel to a horizontal direction relative to the ceiling or floor of the examination room.

The moving carriage 4 is disposed at a lower side of the second guide rail 3 so as to move along the second guide rail 3. The moving carriage 4 is provided with a roller (not shown) that moves along the second guide rail 3.

The post frame 50 is disposed at a lower side of the moving carriage 4 while being fixed to the moving carriage 4. The post frame 50 is provided with a plurality of posts or members. The plurality of posts are connected in a foldable manner to each other so that the post frame 50 may be increased or decreased lengthwise in the up/down direction of the examination room while being fixed to the moving carriage 4. The direction in which the post frame 50 is increased or decreased lengthwise is defined as a third direction D3. Accordingly, the third direction D3 is perpendicular to the first direction D1 and the second direction D2, and substantially parallel to a vertical direction relative to the ceiling or floor of the examination room.

The imaging apparatus 5 is configured to project X-rays toward an object, such as the patient 6. The imaging apparatus 5 may represent an X-ray tube that is typically provided in an X-ray imaging system known in the art. The X-ray tube is provided with an X-ray generating source 13 to generated X-rays, and a collimator 14 to guide the generated X-rays toward the object or patient 6.

A rotating joint 7 is disposed between the imaging apparatus 5 and the post frame 50. The rotating joint 7 is configured to couple the imaging apparatus 5 to the post frame 50 and to support the load acting on the imaging apparatus 5. The rotating joint 7 is provided with a first rotating joint 16 connected to a lower end of the post frame 50, and to a second rotating joint 15 connected to the imaging apparatus 5.

The first rotating joint 16 is provided so as to rotate on a central shaft of the post frame 50 that extends in the upward/downward direction of the examination room. Accordingly, the first rotating joint 16 is rotatable in a plane which is provided perpendicular to the third direction D3. In this case, the direction in which the first rotating joint 16 is rotated is defined as a fifth direction D5. The fifth direction D5 corresponds to a rotation direction of a shaft which is provided in parallel to the third direction D3.

The second rotating joint 15 is provided to rotate in a plane which is perpendicular to the ceiling of the examination room. Accordingly, the second rotating joint 15 rotates in a rotation direction of a shaft which is provided in parallel to the first direction D1 or the second direction D2. In this case, the direction in which the second rotating joint 15 is rotated is defined as a fourth direction D4. The fourth direction D4 corresponds to a rotation direction of a shaft which extends in the first direction D1 or the second direction D2.

The imaging apparatus 5 may be rotated in the fourth direction D4 and the fifth direction D5 while being connected to the rotating joint 7. In addition, the imaging apparatus 5 may linearly move in the first direction D1, the second direction D2 and/or the third direction D3 while being connected to the post frame 50 through the rotating joint 7.

In order to move the imaging apparatus 5 in the first to fifth directions, labeled D1 to D5, respectively, the driving apparatus is provided in a manner known in the art. The driving apparatus may be a motor that is operated by electricity. The driving apparatus may be connected to a power transmission member (not shown) to allow the imaging apparatus 5 to rotate or linearly move in the first to fifth directions, labeled D1 to D5, respectively. The power transmission member may include a belt, a pulley, a chain, a sprocket, and a shaft, and/or any other known type of power transmission mechanism.

The X-ray imaging system 1 is provided with X-ray receiving portions 8, 9, and 10, and further includes a photographing stand 11 and a patient table 20. The X-ray receiving portions 8, 9, and 10 are manufactured to be available to be used with both the photographing stand 11 and the patient table 20. The patient table 20 is movably provided to move relative to the floor of the examination room.

The X-ray receiving portions 8, 9, and 10 are configured to receive the X-rays that are generated from the imaging apparatus 5 and passing through the object or patient 6, and to convert the received X-rays to an electric signal in proportion to the intensity of the received X-rays so that the result of the X-ray photography and examination is diagnosed in real time.

In addition, the X-ray imaging system 1 may further include a high voltage generator 12 to generate a high voltage required to generate the X-rays, with appropriate electrical connectors or channels (not shown), such as wires or wireless, providing the power from the high voltage generator 12 to the imaging apparatus 5 to generate the X-rays.

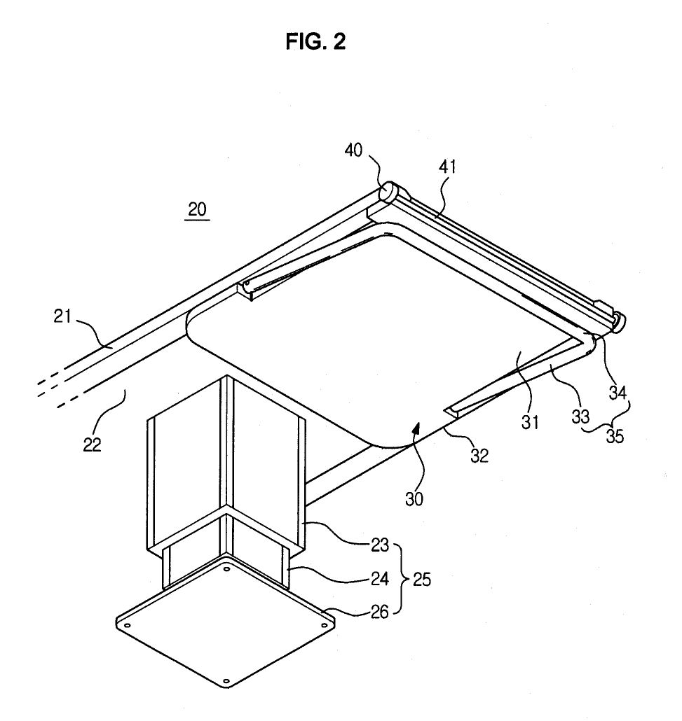

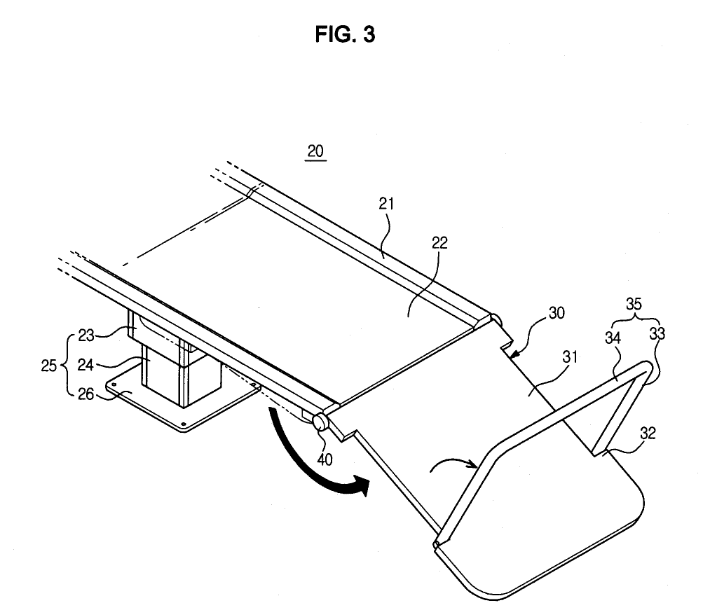

FIG. 2 is an underside perspective view illustrating a folding state of a patient table in accordance with the exemplary embodiment of the present invention. FIG. 3 is a top perspective view illustrating an unfolding state of a patient table in accordance with the exemplary embodiment of the present invention.

Referring to FIGS. 2 and 3, the patient table 20 enables a patient to have an X-ray image obtained while the patient 6 is in a lying position or in a seated position on the patient table 20, and includes a table top 22 and a frame portion 21. A support portion 25 is provided at a lower side of the table top 22 to support the table top 22 on the floor of the examination room.

The support portion 25 is provided with a plurality of supports 23 and 24 that are provided in a foldable and/or extendible manner to adjust the height of the patient table 20. For example, the plurality of supports 23, 24 may have a telescoping configuration, with support 24 slideably movable longitudinally within an interior region of the support 23. In addition, the support portion 25 may include a bottom surface 26 making contact with the ground or floor surface of the examination room while supporting the plurality of supports 23 and 24. The support portion 25 may further include other configurations to drive, adjust, or move the patient table 20 vertically and/or horizontally within the examination room. The shape of the support portion 25, including supports 23, 24 and bottom surface, is not limited to the generally parallelepiped configurations shown in FIGS. 1-10, and the support portion 25 may be provided in other shapes capable of supporting the table top 22 on the ground or floor surface.

Since the X-ray receiving portion 10 is to be provided on the table top 22 to be positioned between the patient 6 and the table top 22, as shown in FIG. 1, the table top 22 needs to be formed using material having a low absorption rate of X-rays such that transmission of X-rays is maximized while minimizing the absorption of X-rays. For example, the table top 22 is formed using any one of or a combination of wood, acryl, and carbon, and/or other known materials which permit sufficient transmission of X-rays therethrough.

In order to reinforce the strength of the table top 22, a frame portion 21 is provided at an edge of the patient table 20. The frame portion 21 is made of, for example, aluminum or other known materials. The frame portion 21 may be provided with equipment for facilitating the examination; for example, with the table top 22 in a sliding configuration with the frame portion 21 which serves as rails for longitudinal movement of the table top 22 with the patient 6 lying thereon. The equipment may include a belt (not shown) to fix an examination region, or a patient handle portion (not shown) to fix the examination region; that is, to position a specific portion of the patient 6, as the examination region, in a selected location where the emitted X-rays will propagate from the X-ray generating source 13.

The patient handle portion is a portion which the subject grips during the examination. The patient handle portion may be optionally detachably installed on the frame portion 21.

A mount portion 30 is rotatably coupled to the table top 22 such that a region of the body of the patient is disposed on the mount portion 30. A stand portion 33 rotatably coupled to the mount portion 30 is provided on the table top 22 such that the X-ray receiving portion 8 can be disposed on the stand portion 33, as shown in FIG. 1. The mount portion 30 is coupled to the frame portion 21 of the table top 22; for example, with a pivoting coupling mechanism.

The mount portion 30 is a portion on which lower portions of the body of the patient 6, such as the entire legs, the lower legs, the feet, and/or other parts of the body, are disposed when the X-ray image is obtained. The mount portion 30 is folded toward a lower side of the table top 22, as shown in FIG. 2, by pivoting, for example, clockwise from the initial position shown in FIG. 3, when the examination is not being performed. Alternatively, the mount portion 30 may be folded by pivoting counterclockwise, depending on the viewing angle toward the mount portion 30 and/or on the implementation of the mount portion 30. The mount portion 30 is made of a carbon plate, fabrics including fibers or leather, and wood, such as medium-density fiberboard (MDF), or any other known materials.

An adjusting unit 40 to adjust a coupling angle between the mount portion 30 and the table top 22 is positioned between the mount portion 30 and the table top 22. The configuration of the adjusting unit 40 will be described in greater detail later.

The mount portion 30 includes an accommodating portion 31 to receive the stand portion 33 in indentations therein, and a damper portion 32 to prevent the stand portion 33 from being inclined at or above a predetermined angle, such as a right angle as shown in FIG. 1. The damper portion 32 protrudes from the sides of the mount portion 30 when compared to the accommodating portion 31 to support the stand portion 33.

The stand portion 33 is pivotally coupled to the mount portion 30. The stand portion 33 is provided to be positioned substantially perpendicular to the mount portion 30. The X-ray receiving portion 8 may be hung on the stand portion 33, for example, using any known type of fasteners such as hooks. Since the X-ray receiving portion 8 receives the X-rays that pass through the patient 6, the X-ray receiving portion 8 needs to be installed while forming an angle of, for example, 90 degrees with respect to the imaging apparatus 5. The stand portion 33 and the mount portion 30 may be hingedly coupled by a joint 41. The adjusting unit 40, installed between the mount portion 30 and the table top 22 to adjust the angle of the stand portion 33, is installed between the stand portion 33 and the mount portion 30. In addition, in order to adjust the angle of the X-rays transmitted to the X-ray receiving portion 8 and/or any of the other X-ray receiving portions 9, 10, the imaging apparatus 5 may pivot as described herein with reference to direction D4 and/or D5, and move in any of the other directions D1, D2, and D3.

The stand assembly 35 includes the stand portion 33 that is coupled to the mount portion 30. The stand portion 33 of the stand assembly 35 is coupled to both sides of the mount portion 30, as shown in FIGS. 2-3. In addition, the stand assembly 35 includes an extension portion 34 that extends between the pair of rods forming the stand portion 33 coupled at the both sides of the mount portion 30. For example, the stand assembly 35 may be provided to have the shape of a “U”. According to the exemplary embodiment of the present invention, the stand assembly 35 may be pivoted so that the extension portion 34 of the stand assembly 35 is positioned substantially adjacent to an upper side of the mount portion 30 while being positioned to face the mount portion 30 when the mount portion 30 is in the folded position shown in FIG. 2.

When the stand portion 33 is not used, the stand portion 33 may be folded by pivoting counterclockwise, or alternatively, clockwise, depending on the viewing angle toward the stand portion 33 and/or on the implementation of the stand portion 33. In a state of having the stand portion 33 folded, the mount portion 30 pivots clockwise to be folded to the lower side of the table top 22, or alternatively the mount portion 30 may pivot counterclockwise, depending on the viewing angle toward the mount portion 30 and/or on the implementation of the mount portion 30. In a state in which the mount portion 30 is folded, the mount portion 30 makes contact with the frame portion 21, as shown in FIG. 2, so that the table top 22 is prevented from being damaged by contact with the mount portion 30 and/or the stand assembly 35 or any part thereof.

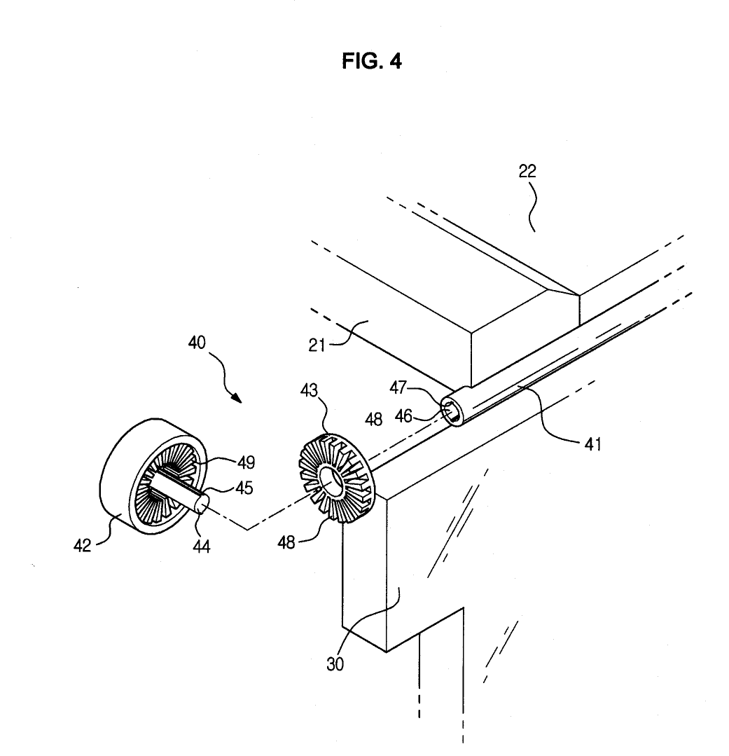

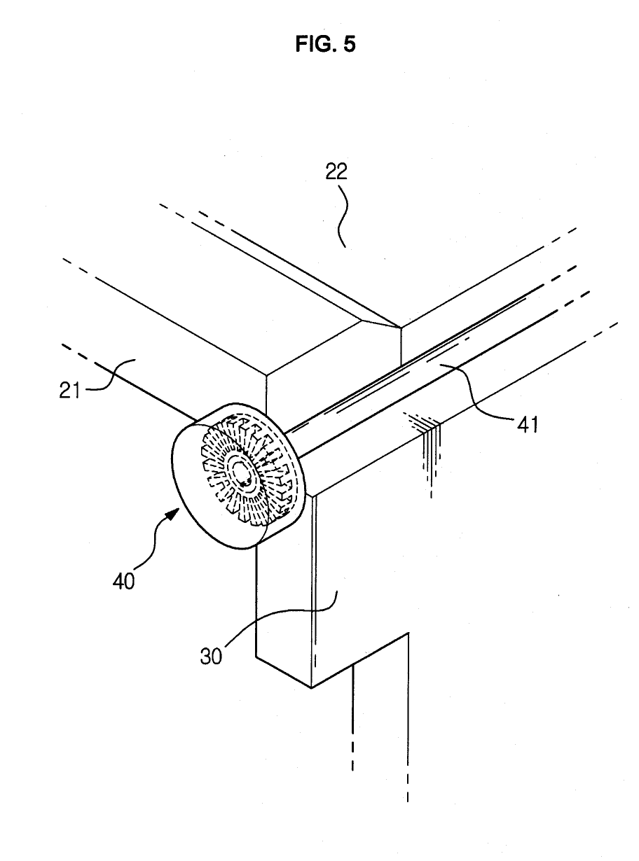

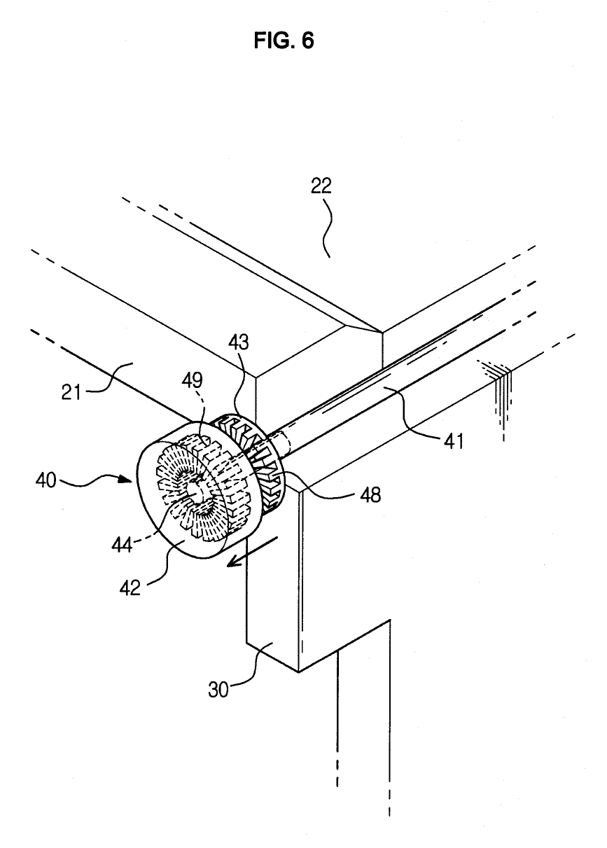

FIG. 4 is an exploded view with parts separated illustrating the adjusting unit 40 in accordance with the exemplary embodiment of the present invention. FIG. 5 is a see-through view illustrating a locking state of the adjusting unit 40 in accordance with the exemplary embodiment of the present invention. FIG. 6 is a view illustrating a releasing state of the adjusting unit 40 in accordance with the exemplary embodiment of the present invention.

The adjusting unit 40 is coupled to a joint 41 that is positioned between the table top 22 and the mount portion 30. The joint 41 connects the table top 22 to the mount portion 30. The adjusting unit 40 includes a body portion 43, which is coupled to the joint 41, and a head portion 42 for engaging the body portion 43. The adjusting unit 40 is positioned between the table top 22 and the mount portion 30. The body portion 43 is inserted into or mounted on the joint 41, and thus the head portion 42 is inserted into or mounted on the joint 41. A central shaft 44 provided in the center of the head portion 42 is inserted into a hole 46 of the joint 41, allowing the adjusting unit 40 to be inserted into the joint 41.

The body portion 43 is provided with at least one installation groove portion 48 having grooves and protrusions, and the head portion 42 is provided with at least one protrusion portion 49, having grooves and protrusions, that is inserted into the installation groove portion 48. The installation groove portion 48 is provided to have a complementary shape relative to the shape of the protrusion portion 49. In a case where the installation groove portion 48 and the protrusion portion 49 are provided with multiple grooves and protrusions, the complementary grooves and protrusions intermesh and provide frictional engagements to have a removably fixed configuration at a variety of rotational orientations, so that the mount portion 30 can be fixed to the table top 22 at a variety of angles, with the frictional engagement between the installation groove portion 48 and the protrusion portion 49 setting the angle depending on their rotational orientation. Accordingly, the more grooves and protrusions that the installation groove potion 48 and the protrusion portion 49 have, the more angles that the mount portion 30 can be adjusted at.

If the protrusion portion 49 is inserted into the installation groove portion 48, the adjusting unit 40 is temporarily fixed, so that the mount portion 30 is temporarily fixed. The state of having the protrusion portion 49 insertedly coupled to the installation groove portion 48 may be defined as a locking state. If the protrusion portion 49 is removed to disengage from the installation groove portion 48, the adjusting unit 40 is able to rotate and the mount portion 30 is able to pivot. The state as such is defined as a releasing state.

The hole 46 of the joint 41 is provided with a locking groove 47 that extends outward of the joint 41. A locking portion 45 corresponding to the locking groove 47 is provided on the central shaft 44 of the head portion 42. In a state when the central shaft 44 of the head portion 42 is coupled to the hole 46 of the joint 41, if the central shaft 44 is slightly rotated, the head portion 42 is prevented from being completely separated from the joint 41 due to the locking portion 45 and the locking groove 47 while maintaining an interval between the head portion 42 and the body portion 43.

The adjusting of the angle of the mount portion 30 of the patient table 20 is as follows. In the locking state in which the protrusion portion 49 of the head portion 42 is inserted into the installation groove portion 48 of the body portion 43, the adjusting unit 40 has a coupling state, and the mount portion 30 is fixed. In a case where the angle between the mount portion 30 and the table top 22 is needed to be adjusted, the head portion 42 of the adjusting unit 40 is pulled out such that the head portion 42 is sufficiently spaced apart from the body portion 43 to disengage the grooves and protrusions thereof, changing the adjusting unit 40 to the releasing state. In the releasing state, the joint 41 coupled to the adjusting unit 40 is rotatable so that the angle of the mount portion 30 is adjusted.

The adjusting unit 40 may be positioned in the area between the mount portion 30 and the stand portion 33 in addition to the coupling area between the mount portion 30 and the table top 22. When the adjusting unit 40 is positioned in the area between the mount portion 30 and the stand portion 33, the angle of the stand portion 33 is freely adjustable.

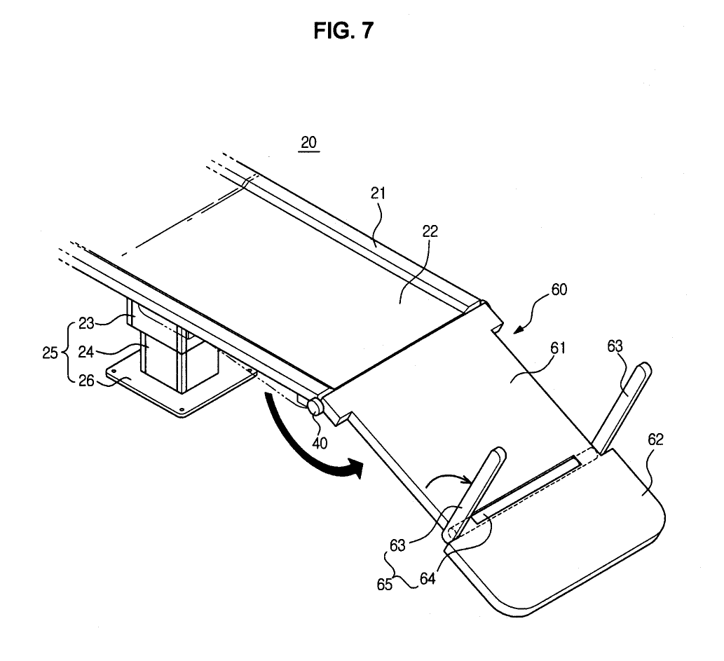

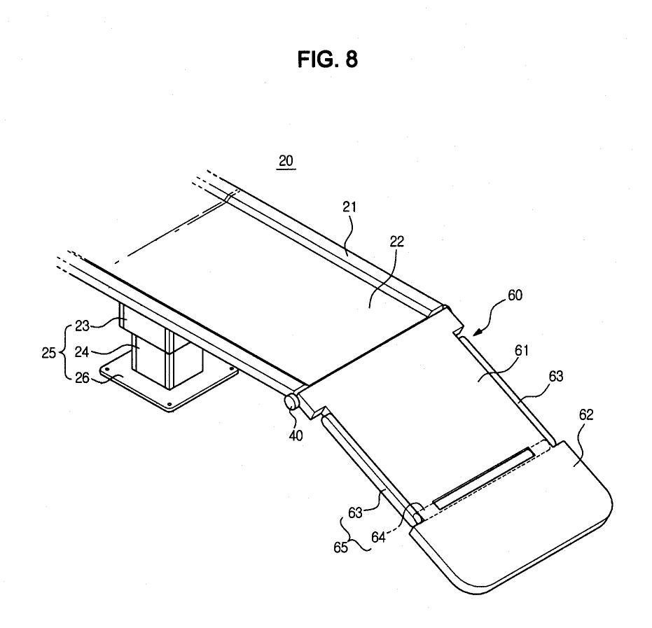

FIG. 7 is a view illustrating an unfolding state of a stand portion 63 of a patient table in accordance with an alternative exemplary embodiment of the present invention. FIG. 8 is a view illustrating a folding state of the stand portion 63 of a patient table in accordance with the alternative exemplary embodiment of the present invention of FIG. 7.

Referring to FIGS. 7 and 8, an extension portion 64 of a stand assembly 65, having the stand portion 63, extends from either side of the stand portion 63, and the extension portion 64 may be integrally formed with the stand portion 63, and attached to or integrally formed with a mount portion 60 while being positioned at a portion of the mount portion 60. The stand assembly 65 is supported by a damper portion 62 of the mount portion 60, so that the X-ray receiving portion 8, shown in FIG. 1, is positioned while being rested on the stand assembly 65.

According to this alternative exemplary embodiment, when the stand assembly 65 is not used, the stand assembly 65 pivots counterclockwise and is folded, or alternatively the stand assembly 65 pivots clockwise, depending on the viewing angle toward the stand assembly 65 and/or on the implementation of the stand assembly 65. In the state of having the stand assembly 65 folded, the mount portion 60 is rotated clockwise and is folded to the lower side of the table top 22. Alternatively, the mount portion 60 is rotated counterclockwise, depending on the viewing angle toward the mount portion 60 and/or on the implementation of the mount portion 60.

In this case, the stand assembly 65 is accommodated while coming into contact with the mount portion 60, in a different manner from the exemplary embodiment of the present invention illustrated with reference to FIGS. 2 and 3. Accordingly, the stand assembly 65 is stored relative to the mount portion 60 without a spatial limitation.

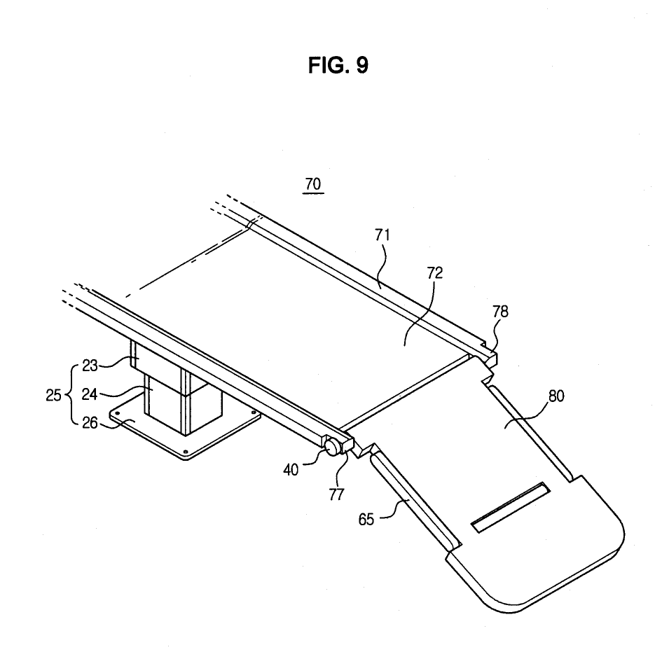

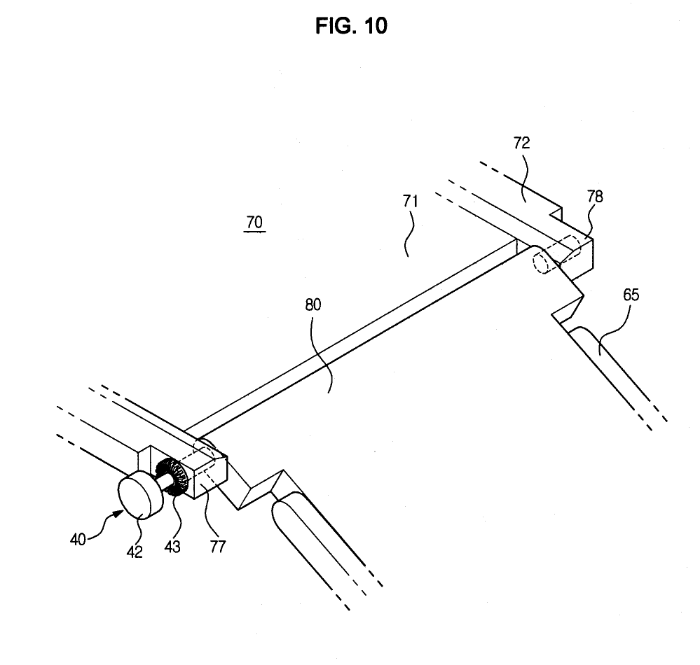

FIG. 9 is a view illustrating an unfolding state of a patient table in accordance with still another alternative exemplary embodiment of the present invention. FIG. 10 is an enlarged view illustrating an assembling portion of the patient table in accordance with the alternative embodiment of the present invention of FIG. 9.

Referring to FIGS. 9 and 10, assembling portions 77 and 78 are provided at a portion of a patient table 70 where the adjusting unit 40 is coupled, so that the adjusting unit 40 is coupled with a joint 41 of the adjusting unit 40 received in the assembling portions 77 and 78. The assembling portion 77 and 78 are formed by extending a portion of the table top 72 of the patient table 70. Alternatively, the assembling portions 77 and 78 may be formed using separate members attached to the table top 72. The adjusting unit 40, passing through the assembling portion 77 provided at one side of the table top 72, is received inward of a mount portion 80 and then received in the assembling portion 78 provided at the other side of the table top 72. According to this embodiment, the mount portion 80 is easily rotated according to the rotation of the adjusting unit 40, in a similar manner described in connection with FIGS. 4-6.

Although a few embodiments of the present invention have been shown and described, it would be appreciated by those skilled in the art that changes may be made in these embodiments without departing from the principles and spirit of the invention, the scope of which is defined in the claims and their equivalents. For example, although the exemplary embodiments of the present invention involved X-rays, the present invention is applicable to other apparatus and methods which use any radiated rays, including X-rays, other rays, and any other known types of radiated rays.

What is claimed is:

1. A patient table comprising:
a table top including material that allows radiated rays to pass therethrough;
a support portion to support the table top;
a mount portion coupled to the table top and having a part of a body of a patient disposed thereon; and
a stand assembly coupled to the mount portion.

2. The patient table of claim 1, further comprising an adjusting unit to adjust a coupling angle between the mount portion and the table top.

3. The patient table of claim 2, wherein the adjusting unit is coupled to a joint between the table top and the mount portion.

4. The patient table of claim 3, wherein the adjusting unit comprises a body portion coupled to the joint and a head portion coupled to the body portion, wherein the body portion is provided with at least one installation groove portion and the head portion is provided with at least one protrusion portion inserted into the at least one installation groove portion.

5. The patient table of claim 4, further comprising a releasing state in which the body portion is spaced apart from the head portion such that the coupling angle between the mount portion and the table top is adjusted, and a locking state at which the body portion is coupled to the head portion such that the mount portion is fixed.

6. The patient table of claim 1, wherein the mount portion pivots in at least one of a clockwise direction or a counterclockwise direction to be folded to a lower side of the table top.

7. The patient table of claim 1, wherein the mount portion comprises an accommodating portion to receive the stand assembly, and a damper portion to prevent the stand assembly from being inclined at or above a predetermined angle.

8. The patient table of claim 7, wherein the stand assembly is folded while pivoting in at least one of a counterclockwise direction or clockwise direction.

9. The patient table of claim 1, wherein the stand assembly comprises a coupling portion that is coupled to the mount portion.

10. The patient table of claim 9, wherein the coupling portion is coupled at each of a pair of sides of the mount portion and comprises an extension portion that extends from the coupling portion coupled at each of the pair of sides of the mount portion.

11. The patient table of claim 10, wherein the extension portion is provided at an upper side of the mount portion while being positioned to face the mount portion.

12. The patient table of claim 10, wherein the extension portion is positioned at one portion of the mount portion as an integral body with the mount portion.

13. The patient table of claim 1, wherein an X-ray receiving portion configured to receive X-rays is disposed on the stand assembly.

14. The patient table of claim 3, wherein the table top comprises an assembling portion, which is provided at each of a pair of sides of the table top and to which the adjusting unit is coupled as a joint of the adjusting unit passes through the assembling portion.

15. The patient table of claim 1, wherein the table top further comprises a frame portion formed at an edge of the table top to reinforce the strength of the table top.

16. The patient table of claim 15, wherein the mount portion is coupled to the frame portion.

17. The patient table of claim 1, wherein the mount portion includes carbon plate, fabrics, or wood.
An imaging system comprising:
a patient table comprising a table top including material that allows radiated rays to pass therethrough, a mount portion coupled to the table top, and a stand assembly coupled to the mount portion;
an imaging apparatus configured to obtain an image of an object by generating the radiated rays, including at least one of X-rays and other rays, and projecting the radiated rays through the object and toward the patient table; and
a radiated ray receiving portion to receive the radiated rays projected from the imaging apparatus.

18. The imaging system of claim 18, wherein the radiated ray receiving portion is disposed on the stand assembly of the patient table, and the stand assembly is fixed while forming a right angle with respect to the mount portion to receive the radiated rays from the imaging apparatus.

19. The imaging system of claim 18, wherein the imaging apparatus is available to pivot to project the radiated rays toward the radiated ray receiving portion.

20. The imaging system of claim 18, wherein the patient table further comprises an adjusting unit configured to adjust a coupling angle between the mount portion and the table top,
wherein the adjusting portion comprises a body portion coupled to a joint between the table top and the mount portion, and a head portion coupled to the body portion, wherein the body portion is provided with at least one installation groove portion, and the head portion is provided with at least one protrusion portion inserted into the at least one installation groove portion.

21. The imaging system of claim 21, wherein the patient table comprises a releasing state at which the body portion is spaced apart from the head portion such that the coupling angle between the mount portion and the table top is adjusted, and a locking state at which the body portion is coupled to the head portion such that the mount portion is fixed.

22. The imaging system of claim 18, wherein the mount portion of the patient table pivots in at least one of a clockwise direction or a counterclockwise direction to be folded to a lower side of the table top.

23. The imaging system of claim 18, wherein the mount portion of the patient table comprises an accommodating portion to receive the stand assembly, and a damper portion to prevent the stand assembly from being inclined at or above a predetermined angle.

24. The imaging system of claim 18, wherein the stand assembly is folded while pivoting in at least one of a counterclockwise direction or a clockwise direction.

25. The imaging system of claim 18, wherein the table top further comprises a frame portion formed at an edge of the table top to reinforce the strength of the table top.
